(19) United States
(12) Patent Application Publication
(10) Pub. No.: US 2025/0134579 A1
(43) Pub. Date: May 1, 2025

(54) METHOD, APPARATUS, AND SYSTEM FOR MANUAL SURGICAL DISSECTION

(71) Applicant: University of Florida Research Foundation, Incorporated, Gainesville, FL (US)

(72) Inventors: Eugene S. Jang, Orinda, CA (US); Mark Thomas Scarborough, Gainesville, FL (US); C. Parker Gibbs, Newberry, FL (US); Andre Spiguel, Gainesville, FL (US)

(21) Appl. No.: 18/835,643

(22) PCT Filed: Feb. 3, 2023
(86) PCT No.: PCT/US2023/061907

§ 371 Date: Aug. 2, 2024

Related U.S. Application Data
(60) Provisional application No. 63/267,947, filed on Feb. 14, 2022.

(51) Int. Cl. A61B 18/14; A61B 18/00; A61B 18/16

(52) U.S. Cl. CPC A61B 18/14; A61B 18/16; A61B 2018/00101; A61B 2018/00601; A61B 2018/00767; A61B 2018/00875

(57) ABSTRACT

Provided herein is a method, apparatus, and system for precise dissection of soft tissue, and more particularly, to the use of a hand-worn device to facilitate dissection while providing tactile feedback to a wearer. Methods include receiving an indication of resistance between at least two leads; establishing a mode of operation based on the indication of resistance; receiving an indication of activation; and providing a current at a voltage and duty cycle based on the mode of operation established to at least one of the at least two leads for electrosurgery. The at least two leads of some embodiments include two electrosurgical devices, where receiving an indication of resistance between the two electrosurgical devices includes receiving an indication of infinite resistance between the two electrosurgical devices, and where the mode of operation is established as bipolar dissection between the two electrosurgical devices.

### CROSS-REFERENCE TO RELATED APPLICATIONS

This application is a national stage entry of PCT Application No. PCT/US2023/061907 filed on Feb. 3, 2023, which claims priority to U.S. Provisional Patent Application 63/267,947, filed on Feb. 14, 2022, the contents of each of which are hereby incorporated by reference in their entirety.

### TECHNOLOGICAL FIELD

An example embodiment of the present disclosure relates to a method, apparatus, and system for precise dissection of soft tissue, and more particularly, to the use of a hand-worn device to facilitate dissection while providing tactile feedback to a wearer.

### BACKGROUND

Surgery is conventionally a challenging process that includes a variety of risk factors which vary based on the specific circumstances of each surgical procedure. Open surgery involving soft tissues often require the precise dissection of soft tissue to ensure optimal surgical outcomes. In surgical procedures involving the removal of benign and malignant lesions, there is an especially small margin for error in terms of the exact location of surgical dissection. In many types of surgery, the determination of where to dissect tissues may be dependent upon properties of the tissue that are not visible distinctions, but may be tactile distinctions. Distinct types of tissue may be distinguishable more so by feel rather than by visual inspection. Conventional surgery offers a limited degree of tactile feedback and precision during dissection.

### BRIEF SUMMARY

Embodiments of the present disclosure provide a method, apparatus, and system for the precise dissection of soft tissue, and more particularly, to the use of a hand-worn device to facilitate dissection while providing tactile feedback to a wearer.

Embodiments provided herein make use of an electrosurgical device, either singular or multiple, which offers the ability to delivery electrical energy via the wearer's fingertip, with heat dissipation properties resulting in minimal heat energy being passed on to the wearer, and with material properties resulting in minimal loss of tactile feedback with use.

Embodiments provided herein include an apparatus including processing circuitry and at least one memory including computer program code, the at least one memory and the computer program code configured to, with the processing circuitry, cause the apparatus to at least: receive an indication of resistance between at least two leads; establish a mode of operation based on the indication of resistance; receive an indication of activation; and provide current at a voltage and duty cycle based on the mode of operation established to at least one of the at least two leads for electrosurgery. The at least two leads of an example embodiment include two electrosurgical devices, where causing the apparatus to receive an indication of resistance between the two electrosurgical devices includes receiving an indication of infinite resistance between the two electrosurgical devices, and where the mode of operation is established as bipolar dissection between the two electrosurgical devices. The current at the voltage and duty cycle includes, in some embodiments, a current at 100% duty cycle at a relatively low root mean square (RMS) voltage, below about 50-volts, based on the mode of operation established as bipolar dissection.

According to some embodiments, the at least two leads include two electrosurgical devices, where causing the apparatus to receive an indication of resistance between the two electrosurgical devices includes causing the apparatus to receive an indication of low resistance between the two electrosurgical devices, and where the mode of operation is established as direct instrument coupling to the two electrosurgical devices. The current at the voltage and duty cycle includes, in some embodiments, a current at 100% duty cycle at a relatively high RMS voltage, above about 100-volts, based on the mode of operation established as direct instrument coupling. According to some embodiments, the at least two leads include two electrosurgical devices and a grounding pad, where causing the apparatus to receive an indication of resistance between at least two leads includes causing the apparatus to receive an indication of conduction only between one of the two electrosurgical devices and the grounding pad, and where the mode of operation is established as monopolar manual dissection using the one of the two electrosurgical devices. The current at the voltage and duty cycle of an example embodiment includes a current at a duty cycle of less than 50% and a relatively high RMS voltage, above about 100-volts.

According to some embodiments, the two leads include two electrosurgical devices, where each of the two electrosurgical devices includes a body, a conductive member, and a lead conducting current to the conductive member. The conductive member is, in some embodiments, embedded in the body, where the body includes a flexible membrane of a thermally insulating closed-cell material. The thermally-insulating closed-cell material includes, in some embodiments, closed cells having a major dimension of no more than two millimeters.

Embodiments provided herein include a method including: receiving an indication of resistance between at least two leads; establishing a mode of operation based on the indication of resistance; receiving an indication of activation; and providing a current at a voltage and duty cycle based on the mode of operation established to at least one of the at least two leads for electrosurgery. The at least two leads of some embodiments include two electrosurgical devices, where receiving an indication of resistance between the two electrosurgical devices includes receiving an indication of infinite resistance between the two electrosurgical devices, and where the mode of operation is established as bipolar dissection between the two electrosurgical devices. The current at the voltage and duty cycle comprises a current at 100% duty cycle and at a relatively low RMS voltage, below about 50-volts, based on the mode of operation established as bipolar dissection.

According to some embodiments, the at least two leads include two electrosurgical devices, where receiving an indication of resistance between the two electrosurgical devices includes receiving an indication of low resistance between the two electrosurgical devices, and where the mode of operation is established as direct instrument coupling to the two electrosurgical devices. The current at the voltage and duty cycle includes, in some embodiments, a current at a 100% duty cycle at a relatively high RMS voltage, above about 100-volts, based on the mode of operation established as direct instrument coupling. According to some embodiments, the at least two leads include two electrosurgical devices and a grounding pad, where receiving an indication of resistance between at least two leads includes receiving an indication of conduction only between one of the two electrosurgical devices and the grounding pad, and where the mode of operation is established as monopolar manual dissection using the one of the two electrosurgical devices. The current at the voltage and duty cycle includes, in some embodiments, a current at a duty cycle of less than 50% and a relatively high RMS voltage, above about 100-volts.

According to some embodiments, the at least two leads include two electrosurgical devices where each of the two electrosurgical devices includes a body, a conductive member, and a lead conducting current to the conductive member. The conductive member of some embodiments is embedded in the body, where the body comprises a flexible membrane of a thermally insulating material. The thermally insulating closed-cell material of some embodiments includes closed cells, and in some embodiments these closed cells have a major dimension of no more than two millimeters.

Embodiments provided herein include an apparatus including: a body including a flexible material; a conductive member including an exposed portion and a portion attached to the body; and an electrical lead, where the body includes a cavity into which a finger of a wearer is received, where the conductive member extends along a portion of the body configured to overlay a pad of the finger of the wearer, where the conductive member receives current through the electrical lead and performs at least one of dissection or electrocauterization along an exposed portion of the conductive member.

The conductive member of an example embodiment is integrally formed with the body, where the exposed portion of the conductive member is no greater than fifty percent of a surface of the conductive member. The conductive member of an example embodiment includes a conductive member cross section having a perimeter, where the conductive member is integrally formed with the body, wherein the body surrounds at least fifty percent of a perimeter of the conductive member cross section. The exposed portion of the conductive member of some embodiments is configurable, where a length of the exposed portion of the conductive member is configurable in at least two lengths.

The flexible material of an example embodiment includes a closed-cell foam material, and wherein cells of the closed-cell foam material have a major dimension no greater than two millimeters.

In an example embodiment, the insulating body is composed of a closed cell foam material. The cells of the closed-cell foam material include, in an example embodiment, an internal pressure lower than atmospheric pressure. The conductive member of an example embodiment has a diameter of less than one millimeter. According to some embodiments, the body is a first body and the flexible material is a first flexible material, the apparatus further including: a second body including a second flexible material; a second conductive member including an exposed portion and a portion attached to the second body, where the second body includes a cavity into which a different finger of a wearer is received, where the exposed portion of the second conducive member is relatively larger than the exposed portion of the first conductive member, where the second conductive member is a grounding pad for the first conductive member.

Embodiments provided herein include a system, the system including: an electrosurgical apparatus including: a body including a flexible material; a conductive member including an exposed portion and a portion attached to the body; and an electrical lead, where the body includes a cavity into which a finger of a wearer is received, where the conductive member extends along a portion of the body configured to overlay a pad of the finger of the wearer, where the conductive member receives current through the electrical lead and performs at least one of dissection or electrocauterization along an exposed portion of the conductive member; and a controller configured to: receive an indication of resistance between at least two leads; establish a mode of operation based on the indication of resistance; receive an indication of activation; and provide current at a voltage and duty cycle based on the mode of operation established to at least one of the at least two leads for electrosurgery in response to the indication of activation.

The controller of some embodiments configured to receive the indication of resistance between the two electrosurgical devices includes the controller configured to receive an indication of infinite resistance between the two electrosurgical devices, and where the mode of operation is established as bipolar dissection between the two electrosurgical devices. The current at the voltage and duty cycles of an example embodiment includes a current at a 100% duty cycle at a relatively low RMS voltage, below about 50-volts, based on the mode of operation established as bipolar dissection.

The at least two leads of an example embodiment include two electrosurgical devices, where the controller configured to receive the indication of resistance between the two electrosurgical devices includes the controller configured to receive an indication of low resistance between the two electrosurgical devices, and where the mode of operation is established as direct instrument coupling to the two electrosurgical devices. According to some embodiments, the current at the voltage and duty cycle includes a current at a 100% duty cycle at a relatively high RMS voltage, above about 100-volts, based on the mode of operation established as direct instrument coupling.

The at least two leads include, in some embodiments, two electrosurgical devices and a grounding pad, where the controller configured to receive the indication of resistance between at least two leads includes the controller configured to receive an indication of conduction only between one of the two electrosurgical devices and the grounding pad, and where the mode of operation is established as monopolar dissection using the one of the two electrosurgical devices. According to some embodiments, the current at the voltage and duty cycle includes a current at a duty cycle of less than 50% and a relatively high RMS voltage, above about 100-volts.

According to some embodiments, the two electrosurgical devices are asymmetric, with one of the devices having a conductive member with a relatively large surface area, allowing the opposing device with a relatively small surface area to act as a monopolar electrocautery device with more effective charge amplification than which can be offered with a traditional grounding pad.

### BRIEF DESCRIPTION OF THE DRAWINGS

Having thus described certain example embodiments in general terms, reference will hereinafter be made to the accompanying drawings, which are not necessarily drawn to scale, and wherein:

FIG. 1 illustrates several views of a monopolar-type dissection tool according to an embodiment of the present disclosure;

FIG. 2 depicts a bipolar-type dissection tool in which two devices are coupled and worn on two separate fingers according to an example embodiment of the present disclosure;

FIG. 3 illustrates a cross-sectional view of a surface of the device in which an electricity-conducting dissection element is attached to the surface of an insulating layer according to an example embodiment of the present disclosure;

FIG. 4 illustrates a cross-sectional view of a surface of a device in which the dissection element is embedded within the insulating surface according to an example embodiment of the present disclosure;

FIG. 5 illustrates a cross-sectional view of the surface of an embodiment of the device in which the insulating layer includes a closed-cell material with pockets of air, highly insulating material, or heat-dissipating material embedded therein according to an example embodiment of the present disclosure;

FIG. 6 illustrates a hybrid two-finger monopolar device whereby one electrode is a thin wire to concentrate current and the other electrode is relatively wider serving as a grounding pad to increase charge density according to an example embodiment of the present disclosure;

FIG. 7 illustrates the thermal insulating properties of a device in which the temperature decreases rapidly as a function of distance across the insulating layer according to an example embodiment of the present disclosure;

FIG. 8 illustrates a device in which the amount of exposed conducting dissecting element may be adjusted during use or a plurality of exposed lengths can be created, allowing for adjustments in the intensity and area of energy delivery according to an example embodiment of the present disclosure;

FIG. 9 illustrates a device with a fuse built into the circuit to act as a fail-safe whereby the fuse prevents the device from functioning after a predetermined amount of energy has been delivered to prompt the use of a new device before the conducting element can fail from thermal changes or plastic deformation according to an example embodiment of the present disclosure;

FIG. 10 illustrates a system by which the electrosurgical unit can alter behavior of the device depending upon the resistance encountered across the circuit, to either allow for direct bipolar dissection or monopolar delivery of energy through an instrument according to an example embodiment of the present disclosure;

FIG. 11 is an example apparatus for implementing methods of operating electrocautery devices according to an example embodiment of the present disclosure; and

FIG. 12 is a flowchart of a method for use of a hand-worn device to facilitate dissection while providing tactile feedback to a wearer according to an example embodiment of the present disclosure.

### DETAILED DESCRIPTION

Some example embodiments of the present disclosure will now be described more fully hereinafter with reference to the accompanying drawings, in which some, but not all embodiments of the invention are shown. Indeed, various embodiments of the disclosure may be embodied in many different forms and should not be construed as limited to the example embodiments set forth herein; rather, these example embodiments are provided so that this disclosure will satisfy applicable legal requirements. Like reference numerals refer to like elements throughout.

Embodiments of the present disclosure relates to a method, apparatus, and system for precise dissection of soft tissue, and more particularly, to the use of a hand-worn device to facilitate dissection while providing tactile feedback to a wearer. Embodiments generally relate to all types of open surgery involving soft tissues in humans and non-human animals including, but not limited to, orthopedic surgery, general surgery, cardiothoracic surgery, breast and obstetric/gynecologic surgery, neurologic surgery, urology, vascular surgery, and plastic and maxillofacial surgery, in which electrocautery may be useful during the course of a surgical procedure. A system as described herein includes a medical device which is worn by a surgeon, such as on a surgeon's fingers, and enables the direct manual dissection of tissues via electrocautery in either a monopolar or bipolar manner.

Throughout the course of any open surgical procedure, the precise dissection of soft tissue is critical to surgical outcomes. In surgical procedures involving the removal of benign and malignant lesions, there is an especially small margin for error in terms of the exact location of surgical dissection. In many types of surgery, the determination of where to dissect tissues is dependent largely upon tactile feedback due to the fact that distinct types of tissue may be distinguishable more so by feel rather than by visual inspection. Conventional surgery offers a limited degree of tactile feedback and precision during dissection.

Traditional methods of dissection include direct application of monopolar electrocautery onto tissues, which can be challenging in terms of precisely defining the depth of penetration as there is little tactile feedback. Further, in deeper cavities or in procedures with heavy bleeding, electrocautery may not function properly due to the presence of fluid. The use of bipolar electrocautery offers the benefit of being more precise and is better suited to work in deeper cavities; however, there is only some indirect tactile feedback. Further, bipolar electrocautery is more time-consuming, and the depth of penetration is not controlled. The use of surgical instruments for direct cutting of tissue or delivery/separation from other tissues to facilitate dissection offers precision and depth control, but lacks substantial tactile feedback.

Embodiments provided herein overcome the issues of conventional electrocautery tools and methods using a device which avoids the shortcomings described above. Embodiments work in concert with existing electrosurgical unit (ESU) systems to deliver radiofrequency (RF) energy in a manner that enables a high degree of tactile feedback, control of resection depth, and precision. According to an example embodiment, a thin, flexible insulating cap is fitted over a surgeon's finger. The insulating cap can be of a selected size from among sizes configured to securely fit on a variety of sizes of hands, and the insulating cap may be worn over a gloved finger. An exposed conductive member serves as the cutting surface as RF energy is delivered to the exposed conductive portion of the conductive member. According to an example embodiment, an insulated wire delivers the current in a monopolar manner via traditional electrosurgical unit interfaces. In such a monopolar embodiment, a single fingertip device is used to divide tissues while an actuator, that can be actuated using a footswitch or finger-actuated button is used to control the on/off and cut/coagulate behavior of the device. The insulating cap worn by the surgeon serves as a thermal insulator to protect the surgeon's fingers during this process. The entire device can be sterilized prior to use in surgery.

According to another example embodiment described herein, two flexible, insulating caps can be worn on two fingers, of the same or opposing hands, for bipolar operation. When used together, these devices can be connected to a traditional electrosurgical unit in a bipolar mode, and the two finger-worn caps serve as elements for the surgical dissection. A footswitch or separate button for the contralateral hand can then be used to control the on/off and cut/coagulate behavior of the device, which allows for manipulation and palpation of tissues prior to surgical dissection. This embodiment can be combined with pre-existing technologies for current modulation in order to allow the bipolar device to seal blood vessels within the tissues. Optionally, a computerized electrosurgical unit enables automatic switching between different modes of electrocautery based on the configuration of electrodes to allow for optimal delivery of energy to carry out the intended type of dissection.

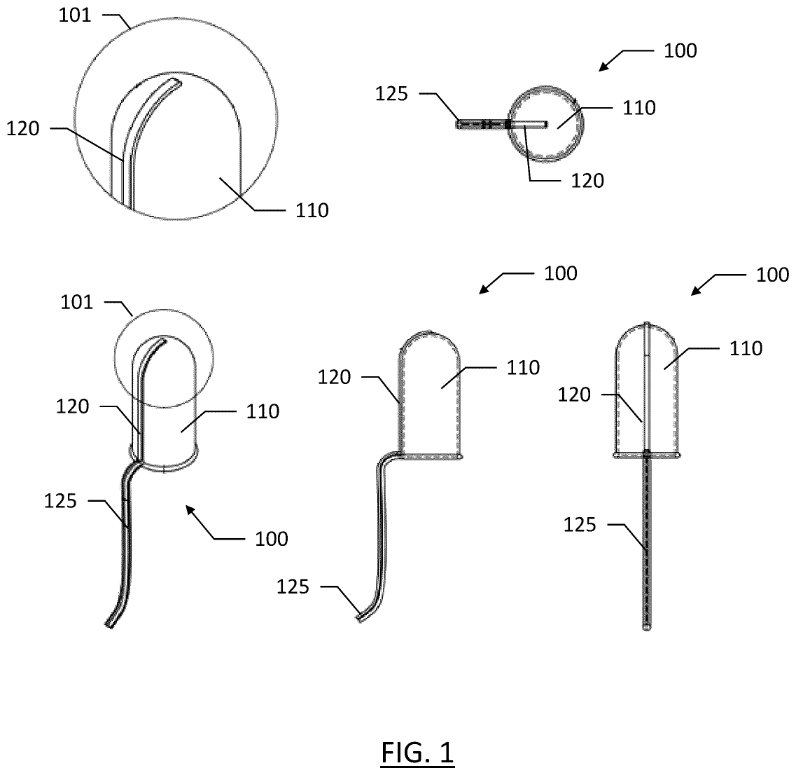

FIG. 1 illustrates several views of an example embodiment of a device provided herein that can be used in a monopolar fashion or in pairs in a bipolar fashion. The device 100 of the illustrated embodiment includes a body 110 which may be of a flexible and relatively thin material that functions as both an electrical and thermal insulator. The thickness of the material is a balance between the insulative properties needed as described further below, and maintaining flexibility to afford substantial tactile feedback between the conductive member cutting element and the finger on which the device is worn. The conductive member 120 is illustrated as a thin, flexible wire that delivers RF energy. Detail circle 101 illustrates the body 110 and the conductive member 120 in greater detail. The conductive member 120 may be formed of tungsten, stainless steel, copper, and other conductive materials or combination of materials. The thermal insulator of the body 110 may include silicone rubber, which can with stand 100-400 degree Celsius operating temperatures that can be encountered during electrocautery. A wire lead 125 conducts electricity from the electrosurgical unit to the conductive member 120 and may be insulated along its length.

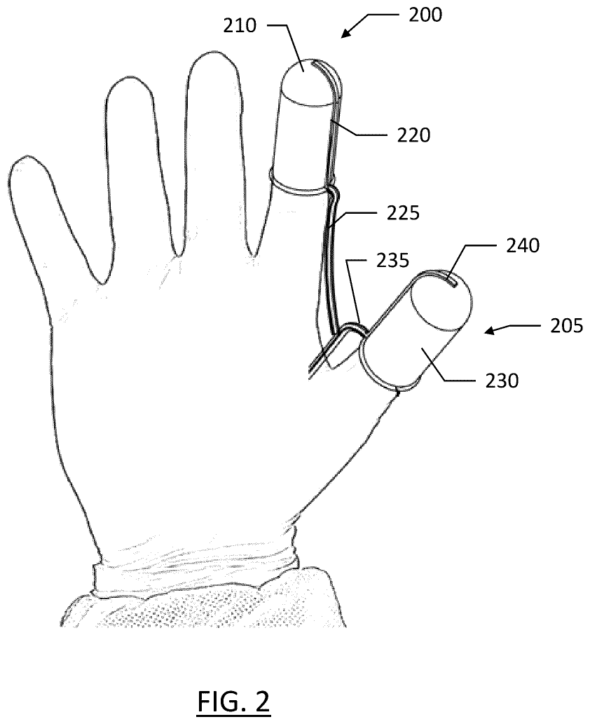

FIG. 2 illustrates an example device in a bipolar configuration in which a first device 200 and a second device 205 are placed on two different fingers of a surgeon's gloved hand. The first device 200 includes a first body 210 and first conductive member 220 while the second device 205 includes a second body 230 and a second conductive member 240. The first device 200 can be connected to an electrosurgical unit via wire lead 225, while the second device 205 can be connected to the electrosurgical unit via wire lead 235 via a bipolar interface of the electrosurgical unit.

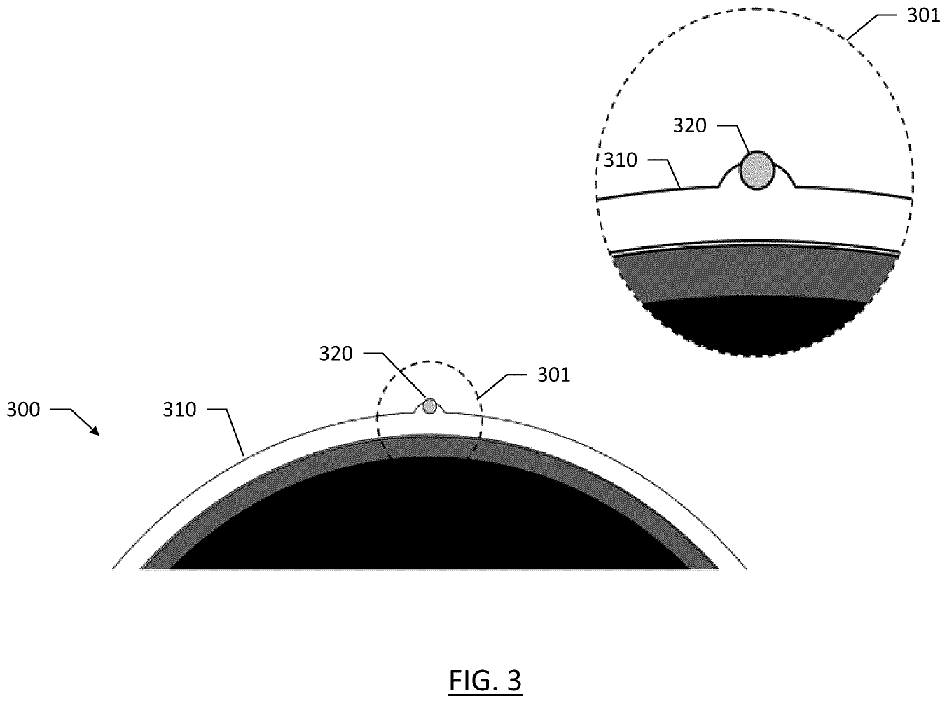

The device of example embodiments can be configured in a number of ways while achieving the desired results. For example, FIG. 3 illustrates a cross-sectional view of a device of the aforementioned embodiments with the cross section taken through the body 310 of the device 300 and through the conductive member 320. Detail circle 301 illustrates the cross section of the conductive member 320 in larger scale. As shown, the conductive member is attached to the body 310, and may be attached through high-temperature stable adhesive, ultrasonic welding, or the like.

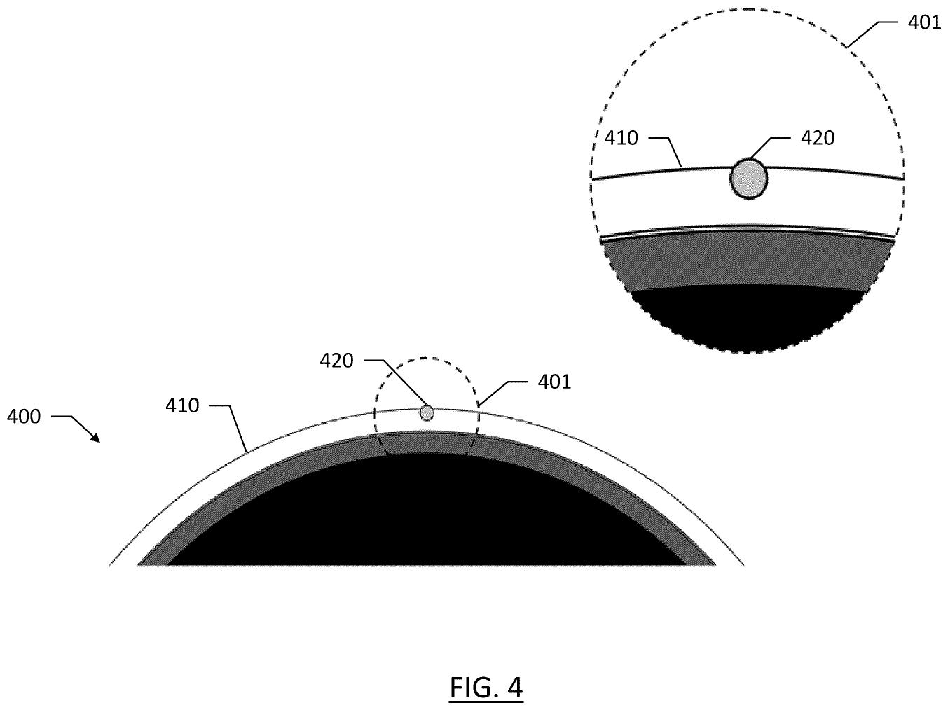

FIG. 4 illustrates a cross-section of another embodiment of a device 400 including device body 410 and conductive member 420. Detail circle 401 illustrates an enlarged view of the cross-section. The conductive member 420 of the embodiment of FIG. 4 is embedded within the body 410. The conductive member 420 may be embedded into the body 410 during a molding or forming process of the body, for example. Embedding the conductive member 420 into the body 410 allows for more consistent thickness of the conductive member 420 and body 410 which results in more consistent and repeatable tactile feedback. Further, embedding the conductive member 420 within the body 410 allows a specified amount of “reveal” or exposure of the conductive member through the surface of the body. By restricting the amount of conductive element exposed, the degree of amplification of current density at the point of electrosurgery is improved.

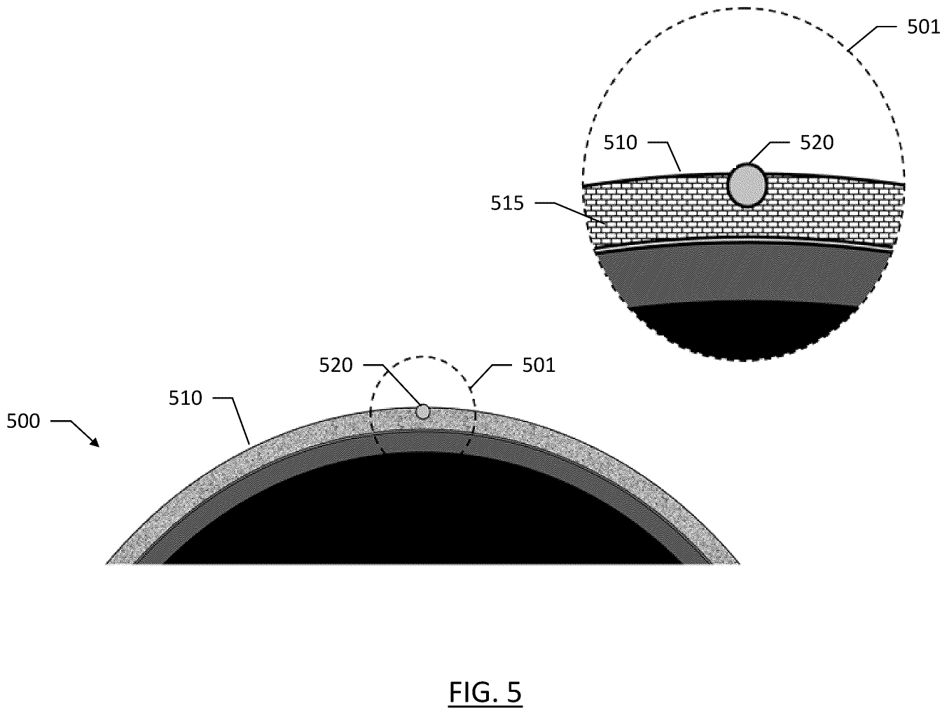

FIG. 5 illustrates a cross section of another embodiment of a device 500 including a device body 510 and a conductive member 520. Detail circle 501 illustrates an enlarged view of the cross-section. The conductive member 520 of FIG. 5 is embedded within an insulative material 515 of the body 510 which may include a thermal insulator such as a closed cell material (e.g. vacuum filled, air filled, etc.), glass fiber material, or other thermally insulative but flexible material. Certain materials, such as a closed-cell material, may enable the insulative material 515 of the body 510 to be relatively thinner while still providing a significant degree of thermal insulation. The closed cells may hold vacuums, such that a pressure within the cells of a closed cell material of example embodiments may have an internal pressure lower than that of atmospheric. For example, a closed cell material may include closed cells having a major dimension (e.g., the largest dimension of the cell) of two millimeters or less, which is around the limit of human perception of two-point discrimination, such that tactile feedback through the material is not adversely affected.

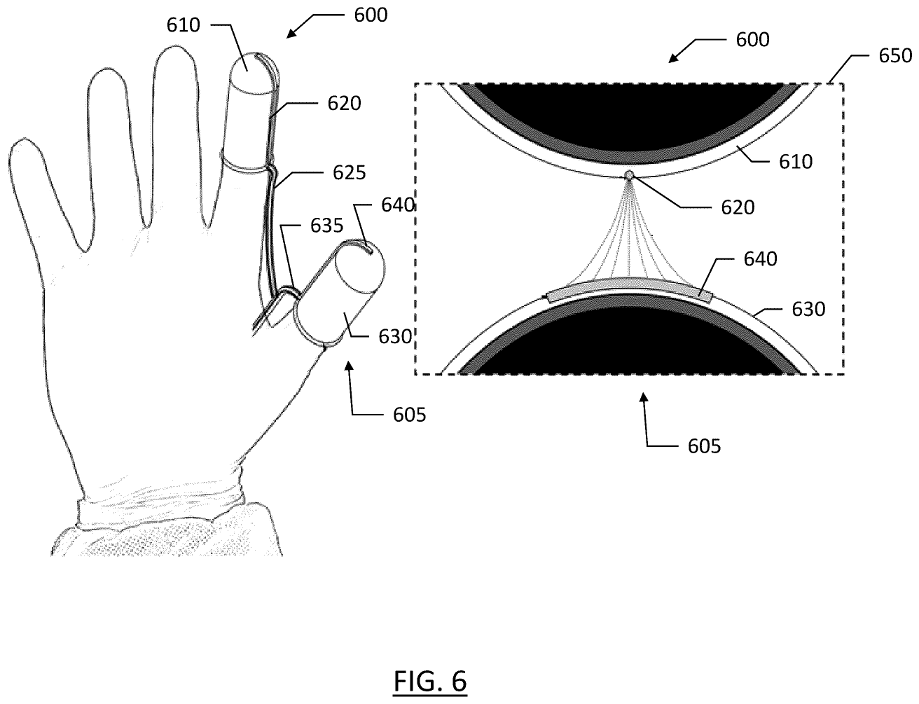

FIG. 6 illustrates another example embodiment of a device as described herein. As illustrated, the embodiment includes a first device 600 having a first body 610, first conductive member 620, and first lead 625 and a second device 605, having a second body 630, second conductive member 640, and second lead 635. The embodiment of FIG. 6 enables a miniaturized version of monopolar electrocautery to occur. Current is carried between a thinner wire (first conductive member 620) and a larger miniature grounding plate (second conductive member 640) as shown in the cross-sectional view 650. The current carried between the relatively thinner wire and grounding plate enables concentration of the current at the point of the thinner wire. The larger grounding plate increases the chances of successful current delivery and depends less on the wires being in close proximity.

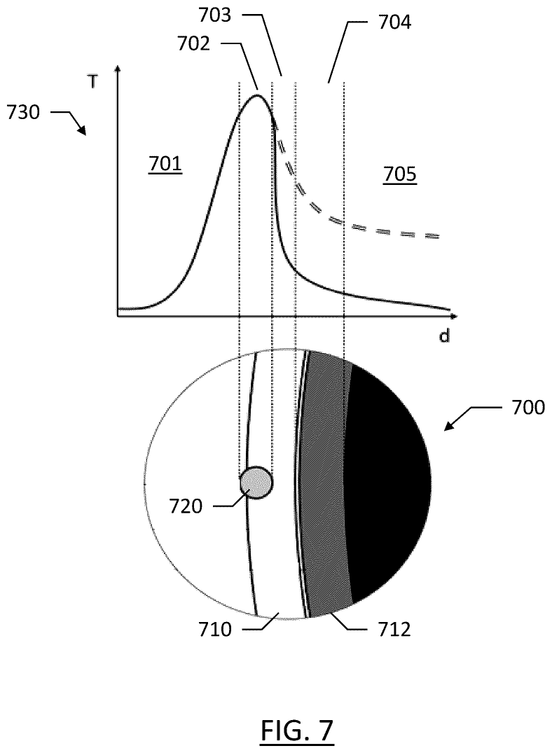

FIG. 7 illustrates a graph 730 demonstrating the temperature across the system as a function of distance away from the surgeon's finger, as mapped onto a cross-section of the device 700. From left-to-right, five zones are illustrated. The first zone 701 illustrates typical quadratic falling-away of energy from the electrode across the tissues being dissected. The second zone 702 depicts the peak temperature experienced at the conducting element 720. The third zone 703 depicts the rapid drop off in temperature (solid line) as a result of the insulating layer of the body 710, as compared to the temperature that would be experienced in the absence of the insulating layer (dashed line). The fourth zone 704 depicts the mild decrease in temperature afforded by the surgical glove 712. The fifth zone 705 illustrates the temperature at the finger of the wearer of the device.

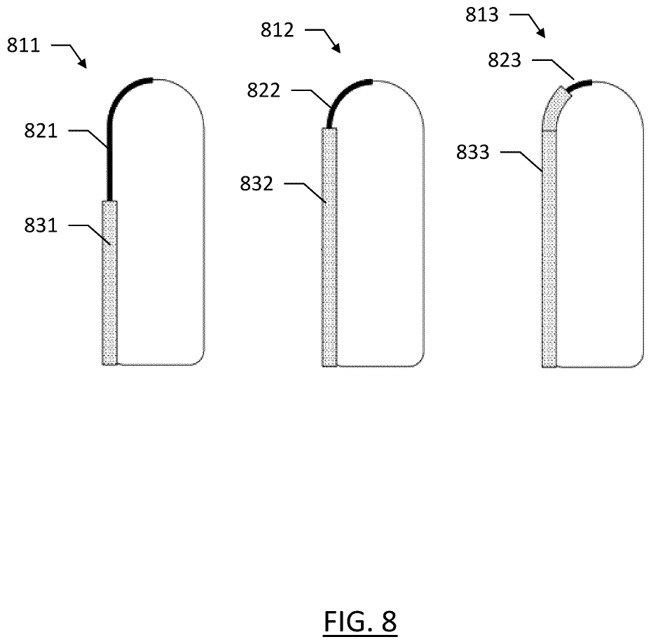

FIG. 8 illustrates three different embodiments of devices as described herein with a variable degree of conductive element exposed, whether as a retractable, adjustable device, or as three separate devices available depending upon the application. A larger area conductive element 821 of device 811 is shown with insulator 831 covering a lower extremity of the conductive element. Device 812 is shown with a greater length of insulator 832 with a shorter conductive element 822 exposed. Device 813 is shown with an even greater length of insulator 833 with a very short conductive element 823 exposed. A larger area of conducting element being exposed allows for faster dissection, while a smaller area shows for a more precise dissection.

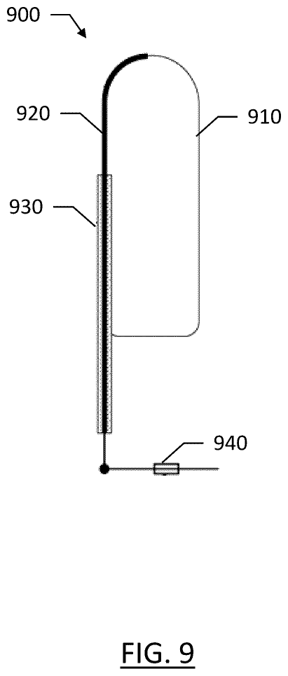

FIG. 9 illustrates a device 900 including a body 910, conductive element 920, and insulator 930. Also illustrated is a fuse 940 built into the connection between the device 900 and the electrosurgical unit. The fuse 940 of an example embodiment is configured to fail before the conducing element 920 itself fails from plastic deformation or thermal degradation. This can be designed with a pre-determined safety factor. As a result, the risk of the conducting element failing during a procedure is reduced.

Embodiments of the device for electrosurgery described herein facilitates tactile feedback not available in prior devices. Traditional monopolar electrocautery (directly onto tissues, onto tissues that are divided by other instruments, or onto the surgeon's finger) or bipolar electrocautery devices do not provide significant tactile feedback. Using embodiments described herein, the entire tissue mass to be dissected can be directly palpated both prior to and during the process of dissection, with multiple fingers. The tactile feedback during the course of dissection enables the surgeon to start and stop the RF energy delivery at will based upon the amount and character of the tissue being dissected. By embedding most of the wire in insulating material, as shown in the embodiment of FIG. 4, and having the material of the body being made of a thin, flexible, and heat resistant material such as silicone, tactile feedback is maximized. Even in the presence of closed-cell materials or other materials that enhance thermal insulation as shown in FIG. 5, provided the closed cells are less than that of the limit of two-point discrimination in the human finger (e.g., around two millimeters), there is minimal reduction in tactile feedback. Further, using a very thin wire (e.g., 0.4 millimeters or 0.015 inches) of a conductive material (e.g., tungsten), there is minimal negative impact on tactile feedback.

Embodiments described herein are additionally more effective in deeper cavities. By directly pressing the device described herein against tissues in deeper cavities, the amount of blood or other fluids surrounding the conductive element is minimized. Because approximately 180-degrees of the conductive member is surrounded by an insulator (e.g., the side attached to the body), exposing only the tissue to the current to be delivered is made easier without the fluid preventing effective electrocautery by diffusing the current delivered. According to an example embodiment, no greater than fifty percent of a surface of the conductive member is exposed. This distinction is substantial as it enables functionality of the devices described herein within environments where substantial fluid exists. The example embodiment of FIG. 4, where the conductive member 420 is embedded within the body 410 is particularly well suited to electrosurgery within environments that may have high fluid content due to the relatively thin area of the conductive member exposed for electrosurgery. Additionally, the surgeon does not need to rely as much on visual inspection in deeper cavities as the tactile feedback provides the surgeon information on the tissue they are working with. The extent of dissection that is possible with embodiments described herein surpasses that which is possible via electrocautery on only visible tissues. Further, a greater portion of the surgical field is accessible to the surgeon.

Typical temperatures generated by electrocautery devices are between 100 and 200 degrees Celsius at the hottest point, proximate the tip of the electrocautery device. Typically, the temperatures are at the lower end of this range. Embodiments described herein allow for maximum thermal insulation between the dissecting wire and the surgeon's finger, to allow for continuous and extended use. Materials that may be appropriate for the body of embodiments described herein include high temperature silicone rubber, which is also flexible and optimizes tactical feedback. Further, high temperature silicone has a high temperature limit of approximately 200-230 degrees Celsius. Silicone rubber can be used indefinitely at working temperatures around 150 degrees Celsius with almost no material property changes and in some formulations can withstand 200 degrees Celsius for 10,000 hours or more. Certain embodiments of silicone rubber can withstand 350 degrees Celsius for short periods of time without adverse effects. Other compatible materials include nitrile and nitrile foams which are used heavily in medical applications and are easily sterilizable. Design considerations, such as the inclusion of air/vacuum/insulator filled cells within the device can be used to maximize thermal insulation, such as in the embodiment of FIG. 5. Embodiments described herein generate less heat than traditional electrocautery devices as the heat generated by an electrical conductor is proportional to a square of the current, and the advantage of current multiplication by having a proportionally thinner conduction area enables a similar surgical effect with less current and thus less heat.

Electrocautery, fundamentally, relies upon the principle of charge density. By having a charge spread out by a large area at the grounding pad (e.g., as shown in FIG. 9), but concentrated at a very small area at the tip of the electrocautery device, a large charge density occurs at the electrocautery device without causing damage at the grounding pad or within intervening tissue. To wit, electrosurgical unit return or grounding pads are typically 100-300 square centimeters, while the tip of an electrocautery device is typically less than ten square millimeters, resulting in a surface charge density magnification of more than 100,000 times. Further, since the concentration of the RF current is reduced with an increase in distance from the active electrode, the current density decreases in quadratic fashion. Combining these two factors (quadratic decrease of charge density and the rate of tissue heating is proportional to the square of current density), the heating occurs in a very localized region at the tip of the electrode.

Embodiments of devices described herein minimize the amount of exposed electrode (e.g., 2-3 millimeters of length of a 0.4 millimeter diameter wire in one embodiment) to further concentrate the charge by an order of magnitude, reducing the amount of current necessary and the heat generated. A range of wire diameters may be used, ideally between 0.2 mm and 2 mm, so as to prevent fatigue failure with narrower wires and loss of tactile feedback with thicker wires. By embedding the conductive element in the body of the device as described herein, and shown particularly in FIG. 4, reducing an exposed length of a conducting element (as shown in FIG. 8), minimizing the diameter of the conducting member, and further concentrating the charge density as shown in FIG. 6, embodiments described herein provide an electrosurgical device that overcomes issues of prior devices while affording valuable tactile feedback to the surgeon.

Finger-mounted electrocautery devices described herein can include a wire or conductive member nearly completely embedded in an insulating layer. The finger of a surgeon serves as an insulator, allowing for very focused application of current while shielding other vital structures. The device of example embodiments, as well as the surgeon's finger and surrounding surgical glove act as a shield against stray current in directions other than the intended plane of surgery.

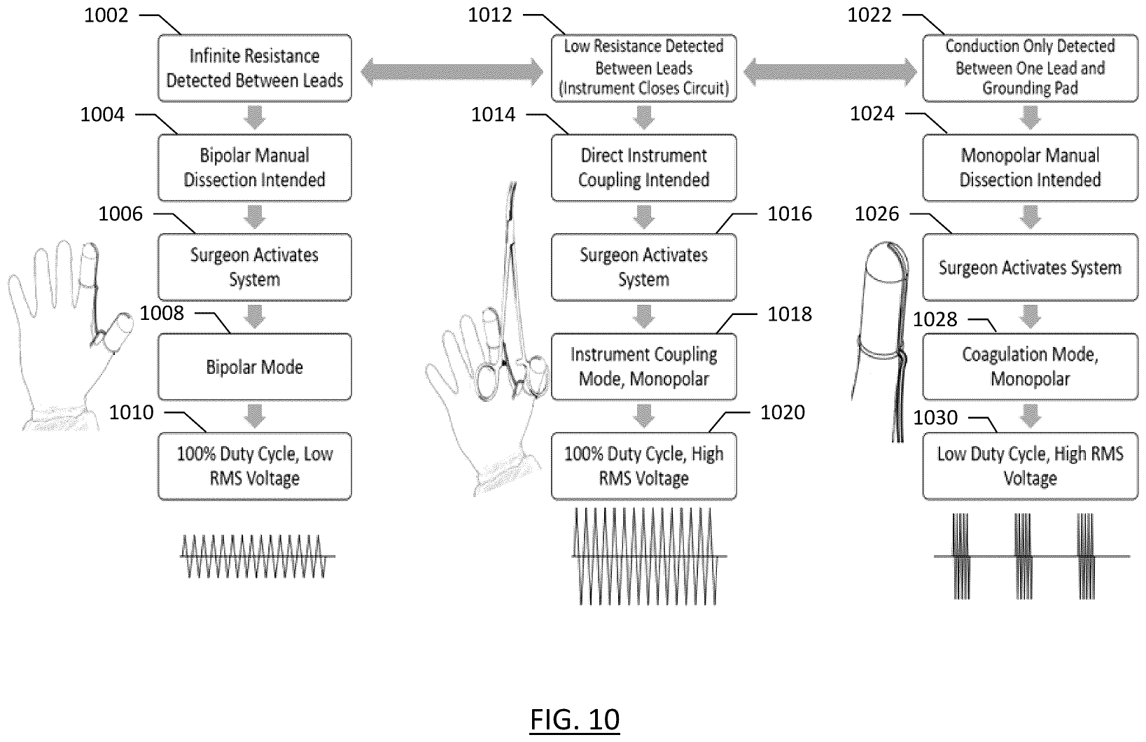

FIG. 10 illustrates an example embodiment in which an electrosurgical unit is computerized and able to adapt to differences in resistance encountered within the system between the three points-two finger dissecting devices and a grounding pad. Embodiments of FIG. 10 may be controlled, such as by a controller described below. If there is an open line detected at the two dissecting devices at 1002 infinite resistance is identified and the system determines that a bipolar dissecting mode is desired at 1004. A surgeon can the activate the system of the electrosurgical unit at 1006, such as using a foot pedal, manual switch, or other means, and energy for bipolar dissection is delivered at 1008, such as a low root mean square (RMS) voltage (e.g., below around 50-volts, such as approximately 40-volts) that is on a 100% duty cycle at 1010, or substantially close to 100% duty cycle. This type of “cut” energy is described as signal with a low “crest factor,” defined as the peak voltage divided by RMS voltage. If the system of embodiments described herein encounters a very low resistance (e.g., less than 1,000 Ohm) between finger dissectors, such as when an instrument (e.g., scissors, scalpel, etc.) closes the circuit at 1012, instrument coupling is detected as intended at 1014. When the electrosurgical system is activated at 1016, a high voltage, 100% duty cycle is delivered to allow the instrument to coagulate and seal vessels with a high efficiency. This energy is still a “cut”-type of low crest factor energy, but delivered at a higher energy level to account for the resistance encountered through the instrument. If a level of resistance equivalent to monopolar electrocautery through a human body is encountered at 1022 between just one electrode and the grounding pad (typical skin contact resistance ranges from 1,000 and 100,000 Ohm), the system converts to a monopolar manual dissection mode at 1024. The monopolar manual dissection mode includes a mode appropriate for monopolar electro cautery, namely a low duty cycle and high RMS voltage for coagulation (e.g., above about 100-volts, such as approximately 200-volts). This type of “coagulation” energy is described as signal with a high crest factor, and has a high peak voltage (e.g., up to 5000-volts) but in a pulsatile fashion. A surgeon can activate the electrosurgical system at 1026 and the monopolar coagulation mode is engaged at 1028. The low duty cycle high RMS voltage current is delivered at 1030. A low duty cycle can include a duty cycle that is less than fifty percent, for example, and may be as low as five percent in some embodiments. Embodiments of the system described herein can optionally include pre-sets that are created for specific instruments and/or specific surgeons in order to account for differences in resistance and to adjust the energy delivery accordingly to achieve the desired surgical result.

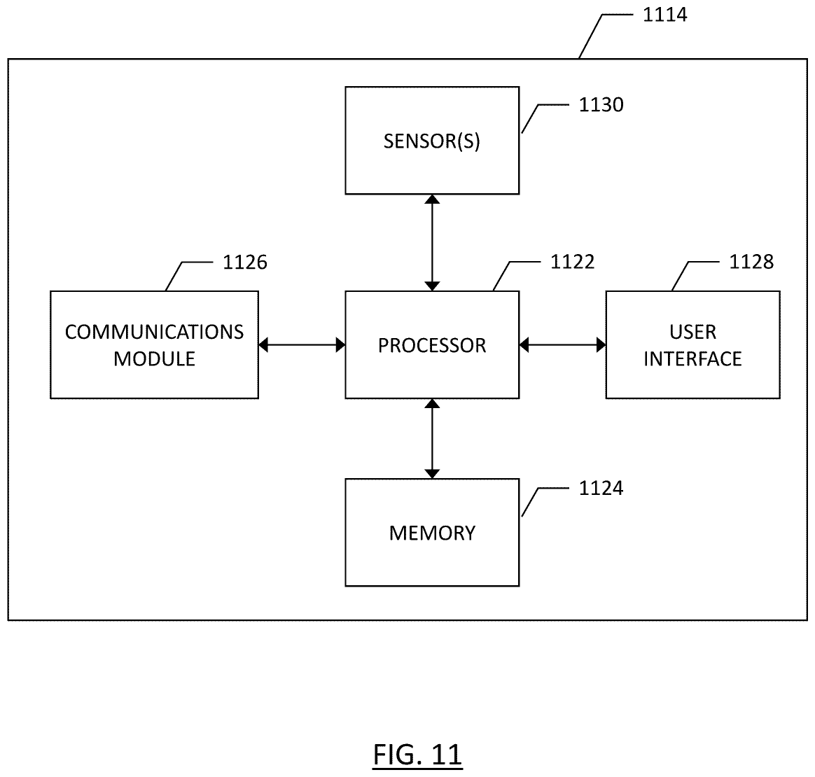

Embodiments of the electrosurgical system described above can be controlled by an apparatus, such as the apparatus of the schematic diagram of FIG. 11 of an example of an apparatus 1114 configured to perform electrosurgical procedures as described herein. The apparatus 1114 may include or otherwise be in communication with a processor 1122, a memory 1124, a communication module 1126, and a user interface 1128. As such, in some embodiments, although devices or elements are shown as being in communication with each other, hereinafter such devices or elements should be considered to be capable of being embodied within the same device or element and thus, devices or elements shown in communication should be understood to alternatively be portions of the same device or element.

In some embodiments, the processor 1122 (and/or co-processors or any other processing circuitry assisting or otherwise associated with the processor) may be in communication with the memory 1124 via a bus for passing information among components of the apparatus. The memory 1124 may include, for example, one or more volatile and/or non-volatile memories. In other words, for example, the memory 1124 may be an electronic storage device (e.g., a computer readable storage medium) comprising gates configured to store data (e.g., bits) that may be retrievable by a machine (e.g., a computing device like the processor). The memory 1124 may be configured to store information, data, content, applications, instructions, or the like for enabling the apparatus 1114 to carry out various functions in accordance with an example embodiment of the present disclosure. For example, the memory 1124 could be configured to buffer input data for processing by the processor 1122. Additionally or alternatively, the memory could be configured to store instructions for execution by the processor.

The processor 1122 may be embodied in a number of different ways. For example, the processor 1122 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor 1122 may include one or more processors configured in tandem via the bus to enable independent execution of instructions, pipelining and/or multithreading. The processor may be embodied as a microcontroller having custom bootloader protection for the firmware from malicious modification in addition to allowing for potential firmware updates.

In an example embodiment, the processor 1122 may be configured to execute instructions stored in the memory 1124 or otherwise accessible to the processor 1122. Alternatively or additionally, the processor 1122 may be configured to execute hard coded functionality. As such, whether configured by hardware or software methods, or by a combination thereof, the processor 1122 may represent an entity (e.g., physically embodied in circuitry) capable of performing operations according to an embodiment of the present invention while configured accordingly. Thus, for example, when the processor 1122 is embodied as an ASIC, FPGA or the like, the processor 1122 may be specifically configured hardware for conducting the operations described herein. Alternatively, as another example, when the processor 1122 is embodied as an executor of software instructions, the instructions may specifically configure the processor 1122 to perform the algorithms and/or operations described herein when the instructions are executed. However, in some cases, the processor 1122 may be a processor of a specific device (e.g., a head-mounted display) configured to employ an embodiment of the present invention by further configuration of the processor 1122 by instructions for performing the algorithms and/or operations described herein. The processor 1122 may include, among other things, a clock, an arithmetic logic unit (ALU) and logic gates configured to support operation of the processor 922. In one embodiment, the processor 922 may also include user interface circuitry configured to control at least some functions of one or more elements of the user interface 1128.

The communication module 1126 may include various components, such as a device or circuitry embodied in either hardware or a combination of hardware and software that is configured to receive and/or transmit data for communicating data between the apparatus 1114 and various other entities, such as a teleradiology system, a database, a medical records system, or the like. In this regard, the communication module 1126 may include, for example, an antenna (or multiple antennas) and supporting hardware and/or software for enabling communications wirelessly. Additionally or alternatively, the communication module 1126 may include the circuitry for interacting with the antenna(s) to cause transmission of signals via the antenna(s) or to handle receipt of signals received via the antenna(s). For example, the communications module 1126 may be configured to communicate wirelessly such as via Wi-Fi (e.g., vehicular Wi-Fi standard 802.11p), Bluetooth, mobile communications standards (e.g., 3G, 4G, or 5G) or other wireless communications techniques. In some instances, the communications module 1126 may alternatively or also support wired communication, which may communicate with a separate transmitting device (not shown). As such, for example, the communications module 1126 may include a communication modem and/or other hardware/software for supporting communication via cable, digital subscriber line (DSL), universal serial bus (USB) or other mechanisms. For example, the communications module 1126 may be configured to communicate via wired communication with other components of a computing device.

The apparatus 1114 described above can be embodied as an electrosurgical unit or controller thereof where the user interface can include the actuation mechanism (e.g., button or foot pedal), while a user interface 1128 can include an indication of the mode of operation and whether current is being delivered or not. Optionally, the user interface 1128 can provide a user the ability to select or configure presets for current delivery and mode of operation according to specific use cases. The communications module 1126 can be employed to receive updates (e.g., software updates, preset updates, etc.) and to communicate with remote devices. The apparatus 1114 can function as a computer controlled electrosurgical unit for refined control of electrosurgical devices described above.

According to example embodiments provided herein, rather than having to deliver tissue via instruments or using electrocautery on the surgeon's finger, the use of these devices can allow for rapid dissection of tissue with fewer interruptions. In traditional techniques, after a portion of tissue is dissected, the surgical bed is reassessed and the surgical instrument and/or finer placed onto a different plane for the process to begin anew. In contrast, embodiments described herein can be used on multiple fingers and potentially on multiple fingers of both hands allowing for rapid transition into different surgical planes and continuation of current surgical planes.

A further aspect of embodiments described herein is the ability to use the smallest incision possible that still allows for effective surgery to reduce morbidity related to larger exposure, such as greater infection risk, more risk of injury to nerves/vessels/other structures, and worse cosmesis. Surgeries performed using embodiments described herein can be performed using relatively smaller exposures than required for surgery requiring direct visualization.

Having both monopolar and bipolar options for the devices of example embodiments, the options available to the surgeons increase in terms of available modes of surgical dissection. In monopolar operation, it is possible to seamlessly transition back and forth between blunt and sharp dissection by using the same finger for both processes and a switch (e.g., user interface 1128) to turn the electrode on and off. In bipolar operation, entire stalks or bulks of tissue can be dissected continuously without interruption, particularly if combined with current modulation techniques that can be employed by the electrosurgical unit of example embodiments.

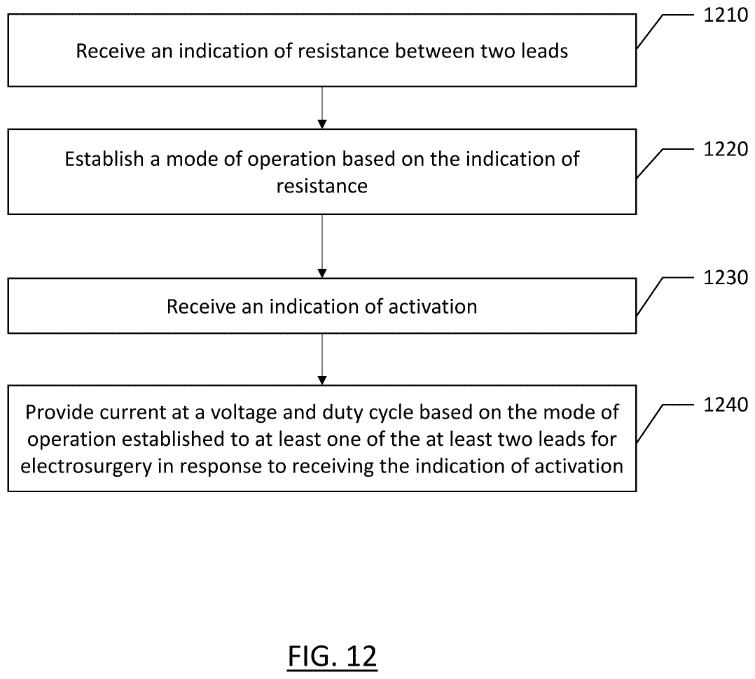

FIG. 12 illustrates a flowchart of a method according to an example embodiment of the disclosure. It will be understood that each block of the flowchart, and combinations of blocks in the flowchart, may be implemented by various means, such as hardware, firmware, processor, circuitry, and/or other devices associated with execution of software including one or more computer program instructions. For example, one or more of the procedures described above may be embodied by computer program instructions. In this regard, the computer program instructions which embody the procedures described above may be stored by the memory 1124 of an apparatus employing an embodiment of the present invention and executed by the processor 1122 of the apparatus. As will be appreciated, any such computer program instructions may be loaded onto a computer or other programmable apparatus (e.g., hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

According to the flow chart of FIG. 12, an indication of resistance is received between two leads at 1210. These two leads may include, for example, two electrosurgical devices as described above. A mode of operation is established based on the indication of resistance at 1220. The mode of operation identifies how the two leads or electrosurgical devices will be used for electrosurgery. An indication of activation is received at 1230. This indication may be received from a surgeon, for example, by activating a foot switch or pressing a button. Current at a voltage and duty cycle are provided to at least one of the two leads for electrosurgery based on the mode of operation established in response to receiving the indication of activation at 1240.

In an example embodiment, an apparatus for performing the method of FIG. 12 above may comprise a processor (e.g., the processor 1122) configured to perform some or each of the operations (1210-1140) described above. The processor may, for example, be configured to perform the operations (1210-1240) by performing hardware implemented logical functions, executing stored instructions, or executing algorithms for performing each of the operations. Alternatively, the apparatus may comprise means for performing each of the operations described above. In this regard, according to an example embodiment, examples of means for performing operations 1210-1240 may comprise, for example, the processor 1122 and/or a device or circuit for executing instructions or executing an algorithm for processing information as described above.

Statement 1: An example apparatus includes: a body comprising a flexible material; a conductive member comprising an exposed portion and a portion attached to the body; and an electrical lead, wherein the body comprises a cavity into which a finger of a wearer is received, wherein the conductive member extends along a portion of the body configured to overlay a pad of the finger of the wearer, wherein the conductive member receives current through the electrical lead and performs at least one of dissection or electrocauterization along an exposed portion of the conductive member.

Statement 2: The apparatus of Statement 1, wherein the flexible material comprises a closed-cell foam material, and wherein cells of the closed-cell foam material have a major dimension no greater than two millimeters.

Statement 3: The apparatus of Statement 2, wherein the cells of the closed-cell foam material comprise an internal pressure lower than atmospheric pressure.

Statement 4: The apparatus of Statement 1, wherein the conductive member comprises a diameter of less than two millimeters.

Statement 5: The apparatus of Statement 1, wherein the conductive member is integrally formed with the body, wherein the exposed portion of the conductive member is no greater than fifty percent of a surface of the conductive member.

Statement 6: The apparatus of Statement 1, wherein the conductive member comprises a conductive member cross section having a perimeter, wherein the conductive member is integrally formed with the body, wherein the body surrounds at least fifty percent of a perimeter of the conductive member cross section.

Statement 7: The apparatus of Statement 1, wherein the exposed portion of the conductive member is configurable, wherein a length of the exposed portion of the conductive member is configurable in at least two lengths.

Statement 8: The apparatus of Statement 1, wherein the body is a first body, the conductive member is a first conductive member, and the flexible material is a first flexible material, the apparatus further comprising: a second body comprising a second flexible material; and a second conductive member comprising an exposed portion and a portion attached to the second body; wherein the second body comprises a cavity into which a different finger of a wearer is received, wherein the exposed portion of the second conductive member is relatively larger than the exposed portion of the first conductive member, wherein the second conductive member is a grounding pad for the first conductive member to focus charge density of the first conductive member.

Statement 9: A system comprising: an electrosurgical apparatus comprising: a body comprising a flexible material; a conductive member comprising an exposed portion and a portion attached to the body; and an electrical lead, wherein the body comprises a cavity into which a finger of a wearer is received, wherein the conductive member extends along a portion of the body configured to overlay a pad of the finger of the wearer, wherein the conductive member receives current through the conductive member and performs at least one of dissection or electrocauterization along an exposed portion of the conductive member; and a controller configured to: receive an indication of resistance between at least two leads; establish a mode of operation based on the indication of resistance; receive an indication of activation; and provide current at a voltage and duty cycle based on the mode of operation established to at least one of the at least two leads for electrosurgery in response to receiving the indication of activation.

Statement 10: The system of Statement 9, wherein the at least two leads comprise two electrosurgical devices, the controller configured to receive the indication of resistance between the two electrosurgical devices comprises the controller configured to receive an indication of infinite resistance between the at least two leads, and wherein the mode of operation is established as bipolar dissection between the two electrosurgical devices.

Statement 11: The system of Statement 10, wherein the current at the voltage and duty cycle comprise a current at a 100% duty cycle at a relatively low RMS voltage, below about 50-volts, based on the mode of operation established as bipolar dissection.

Statement 12: The system of Statement 9, wherein the at least two leads comprise two electrosurgical devices, wherein the controller configured to receive the indication of resistance between the two electrosurgical devices comprises the controller configured to receive an indication of low resistance between the two electrosurgical devices, and wherein the mode of operation is established as direct instrument coupling to the two electrosurgical devices.

Statement 13: The system of Statement 12, wherein the current at the voltage and duty cycle comprise a current at a 100% duty cycle at a relatively high RMS voltage, above about 100-volts, based on the mode of operation established as direct instrument coupling.

Statement 14: The system of Statement 9, wherein the at least two leads comprise two electrosurgical devices and a grounding pad, wherein the controller configured to receive the indication of resistance between at least two leads comprises the controller configured to receive an indication of conduction only between one of the two electrosurgical devices and the grounding pad, and wherein the mode of operation is established as monopolar manual dissection using the one of the two electrosurgical devices.

Statement 15: The system of Statement 14, wherein the current at the voltage and duty cycle comprise a current at a duty cycle of less than 50% and a relatively high RMS voltage, above about 100-volts.

In some embodiments, certain ones of the operations above may be modified or further amplified. Furthermore, in some embodiments, additional optional operations may be included. Modifications, additions, or amplifications to the operations above may be performed in any order and in any combination.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. An apparatus comprising processing circuitry and at least one memory including computer program code, the at least one memory and the computer program code configured to, with the processing circuitry, cause the apparatus to at least:
receive an indication of resistance between at least two leads;
establish a mode of operation based on the indication of resistance;
receive an indication of activation; and
provide current at a voltage and duty cycle based on the mode of operation established to at least one of the at least two leads for electrosurgery in response to the indication of activation.

2. The apparatus of claim 1, wherein the at least two leads comprise two electrosurgical devices,
wherein causing the apparatus to receive the indication of resistance between the two electrosurgical devices comprises causing the apparatus to receive an indication of infinite resistance between the two electrosurgical devices, and
wherein the mode of operation is established as bipolar dissection between the two electrosurgical devices.

3. The apparatus of claim 2, wherein the current at the voltage and duty cycle comprises a current at 100% duty cycle at a relatively low RMS voltage, below about 50-volts, based on the mode of operation established as bipolar dissection.

4. The apparatus of claim 1, wherein the at least two leads comprise two electrosurgical devices,
wherein causing the apparatus to receive the indication of resistance between the two electrosurgical devices comprises causing the apparatus to receive an indication of low resistance between the two electrosurgical devices, and
wherein the mode of operation is established as direct instrument coupling to the two electrosurgical devices.

5. The apparatus of claim 4, wherein the current at the voltage and duty cycle comprises a current at 100% duty cycle at a relatively high RMS voltage, above about 100-volts, based on the mode of operation established as direct instrument coupling.

6. The apparatus of claim 1, wherein the at least two leads comprise two electrosurgical devices and a grounding pad,
wherein causing the apparatus to receive the indication of resistance between at least two leads comprises causing the apparatus to receive an indication of conduction only between one of the two electrosurgical devices and the grounding pad, and
wherein the mode of operation is established as monopolar manual dissection using the one of the two electrosurgical devices.

7. The apparatus of claim 6, wherein the current at the voltage and duty cycle comprise a current at a duty cycle of less than 50% and a relatively high RMS voltage, above about 100-volts.

8. The apparatus of claim 1, wherein the at least two leads comprise two electrosurgical devices, where each of the two electrosurgical devices includes a body, a conductive member, and a lead conducting current to the conductive member.

9. The apparatus of claim 8, wherein the conductive member is embedded in the body, wherein the body comprises a flexible membrane of a thermally insulating material.

10. The apparatus of claim 9, wherein the thermally insulating material comprises a closed-cell material, closed cells of the closed-cell material having a major dimension of no more than two millimeters.

11. A method comprising:
receiving an indication of resistance between at least two leads;
establishing a mode of operation based on the indication of resistance;
receiving an indication of activation; and
providing current at a voltage and duty cycle based on the mode of operation established to at least one of the at least two leads for electrosurgery in response to receiving the indication of activation.

12. The method of claim 11, wherein the at least two leads comprise two electrosurgical devices,
wherein receiving the indication of resistance between the two electrosurgical devices comprises receiving an indication of infinite resistance between the two electrosurgical devices, and
wherein the mode of operation is established as bipolar dissection between the two electrosurgical devices.

13. The method of claim 12, wherein the current at the voltage and duty cycle comprises a current at a 100% duty cycle at a relatively low RMS voltage, below about 50-volts, based on the mode of operation established as bipolar dissection.

14. The method of claim 11, wherein the at least two leads comprise two electrosurgical devices,
wherein receiving the indication of resistance between the two electrosurgical devices comprises receiving an indication of low resistance between the two electrosurgical devices, and
wherein the mode of operation is established as direct instrument coupling to the two electrosurgical devices.

15. The method of claim 14, wherein the current at the voltage and duty cycle comprise a current at a 100% duty cycle at a relatively high RMS voltage, above about 100-volts, based on the mode of operation established as direct instrument coupling.

16. The method of claim 11, wherein the at least two leads comprise two electrosurgical devices and a grounding pad,
wherein receiving the indication of resistance between at least two leads comprises receiving an indication of conduction only between one of the two electrosurgical devices and the grounding pad, and
wherein the mode of operation is established as monopolar manual dissection using the one of the two electrosurgical devices.

17. The method of claim 16, wherein the current at the voltage and duty cycle comprises a current at a duty cycle of less than 50% and a relatively high RMS voltage, above about 100-volts.

18. The method of claim 11, wherein the at least two leads comprise two electrosurgical devices, wherein each of the two electrosurgical devices includes a body, a conductive member, and a lead conducting current to the conductive member.

19. The method of claim 18, wherein the conductive member is embedded in the body, wherein the body comprises a flexible membrane of a thermally insulating closed-cell material.

20. The method of claim 19, wherein the thermally insulating closed-cell material comprises closed cells having a major dimension of no more than two millimeters.
